SEQUENCE LISTING (20 sequences)

SEQ ID NO: 1 - DNA, 24 bp, Artificial sequence
  Primer example 1
agggatgccatgcttggagtttcc

SEQ ID NO: 2 - DNA, 22 bp, Artificial sequence
  Primer example 1
ccaattgccctcatccccatcc

SEQ ID NO: 3 - DNA, 30 bp, Artificial sequence
  Primer La2
ccgatctctccaggccaagctttcctctac

SEQ ID NO: 4 - DNA, 30 bp, Artificial sequence
  Primer La3
gtagaggaaagcgcggcctggagagatcgg

SEQ ID NO: 5 - DNA, 27 bp, Artificial sequence
  Primer La4
acaatgaccctcaagctgccctctacg

SEQ ID NO: 6 - DNA, 27 bp, Artificial sequence
  Primer La5
acaatgacccacgtgctgccctctacg

SEQ ID NO: 7 - DNA, 26 bp, Artificial sequence
  Primer La6
gggagcggggatctggtaccaatcgg

SEQ ID NO: 8 - DNA, 33 bp, Artificial sequence
  Primer La7
ggagggagcggggatgcgataccaatcggcgag

SEQ ID NO: 9 - DNA, 29 bp, Artificial sequence
  Primer La8
ttactgagcctcaaacggttgatcgtctc

SEQ ID NO: 10 - DNA, 29 bp, Artificial sequence
  Primer La9
ttactgagccgcgcacggttgatcgtctc

SEQ ID NO: 11 - DNA, 27 bp, Artificial sequence
  Primer La10
ggtcgatgagagcctgcaggtcggctt

SEQ ID NO: 12 - DNA, 27 bp, Artificial sequence
  Primer La11
ggtcgatgagagcgcggaggtcggctt

SEQ ID NO: 13 - DNA, 36 bp, Artificial sequence
  Primer BamHI-fwd
cgtggatccttcaccatgttcaagaacctcctctcg

SEQ ID NO: 14 - DNA, 21 bp, Artificial sequence
  Primer 2801-rev
ggattgattgtctaccgccag

SEQ ID NO: 15 - DNA, 26 bp, Artificial sequence
  Primer example 2 a)
gtttcccgactggaaagcgggcagtg

SEQ ID NO: 16 - DNA, 23 bp, Artificial sequence
  Primer example 2 a)
caccgatagggaagaggccctcg

SEQ ID NO: 17 - DNA, 21 bp, Artificial sequence
  Primer example 2 b)
gagagtcagtcagcttcatgt

SEQ ID NO: 18 - DNA, 22 bp, Artificial sequence
  Primer example 2 b)
gcttctgcgttctgatttaatc

SEQ ID NO: 19 - DNA, 24 bp, Artificial sequence
  Primer example 3
agggatgccatgcttggagtttcc

SEQ ID NO: 20 - DNA, 22 bp, Artificial sequence
  Primer example 3
ccaattgccctcatccccatcc

(19) United States
(12) Patent Application Publication
(10) Pub. No.: US 2004/0152094 A1
(43) Pub. Date: Aug. 5, 2004

(54) Method for producing recombined polynucleotides

(76) Inventors: Steffen Danielsen, Copenhagen (DE); Torben Vedel Borchert, Birkerod (DK)

Correspondence Address: NOVOZYMES NORTH AMERICA, INC., 500 FIFTH AVENUE, SUITE 1600, NEW YORK, NY, 10110, US

(21) Appl. No.: 10/477,376

(22) PCT Filed: May 7, 2002
(86) PCT No.: PCT/DK02/00294

(30) Foreign Application Priority Data
May 10, 2001 (DK) PA200100743

(51) Int. Cl. C12Q 1/68; C12P 19/34

(52) U.S. Cl. 435/6; 435/91.2

(57) ABSTRACT

The present invention concerns briefly a method for producing recombined polynucleotides by utilizing nucleotides or nucleotide analogues not normally present in naturally occurring polynucleotides, wherein the sugar-base bonds are cleavable, or from which the base-moiety can be cleaved, thus generating so-called AP-sites. These AP-sites may be used for generating random sized polynucleotide fragments for use in a shuffling procedure.

### FIELD OF THE INVENTION

The present invention relates to optimizing DNA sequences in order to alter one or more properties of a protein of interest by generating recombined polynucleotides encoding proteins of interest. This is achieved through the use of a so-called gene- or DNA shuffling technique to create large libraries of genes, expressing said library of genes in a suitable expression system and screening the expressed encoded proteins for specific characteristics in order to identify the proteins that exhibit the desired altered property. The present invention also relates to altering one or more properties of regulatory polynucleotide elements such as promoters, transcription terminators, enhancers, activators etc.

### BACKGROUND OF THE INVENTION

It is generally found that similar proteins having an identical activity may exhibit a certain nucleotide sequence variation in the encoding genes between genera and even between members of the same species. This natural genetic diversity among genes coding for proteins having basically the same bioactivity has evolved in Nature through time and reflects a natural optimization of the proteins coded for in respect of the particular micro-environment or“niche” of the individual organisms.

Naturally occurring bioactive molecules are not optimized for the various uses to which they are put by mankind, certainly not when they are used for industrial purposes.

It has therefore for quite a while been an interest of Industry to modify and select or screen for bioactive polypeptides or proteins that exhibit optimal properties in respect of the use to which it is intended or the micro-environment in which it is going to be used.

This optimization has classically been done by screening polypeptides of natural sources, or by use of mutagenesis. For instance, within the technical field of enzymes for use in detergents, the washing and/or dishwashing performance of naturally occurring proteases, lipases, amylases and cellulases have been improved significantly, by in vitro modifications of the enzymes.

In most cases these improvements have been obtained by site-directed mutagenesis resulting in substitution, deletion or insertion of specific amino acid residues which have been chosen either on the basis of their type or on the basis of their location in the secondary or tertiary structure of the mature enzyme (see for instance U.S. Pat. No. 4,518,584). In this manner the preparation of novel polypeptide variants and mutants, such as novel modified enzymes with altered characteristics, e.g. specific activity, substrate specificity, thermal-, pH-, and salt stability, pH-optimum, pI, Kₘ, Vₘₐₓ etc.

Weber et al., (1983), Nucleic Acids Research, vol.11, 5661, describes a method for modifying genes by in vivo recombination of two homologous genes. In WO 97/07205 a method is described whereby polypeptide variants are prepared by shuffling different nucleotide sequences of homologous DNA sequences by in vivo recombination.

A method for the shuffling of homologous DNA sequences has been described by Stemmer et al. in WO 95/22625. An important step in this method is to cleave or fragment the homologous template double-stranded polynucleotide into random fragments of a desired size by treatment with DNase I followed by homologously reassembling of the fragments into full-length genes.

WO 98/01581 relates to a method of blocking or interrupting the DNA-synthesis process at random positions by utilization of UV-light, DNA adducts, or DNA binding proteins.

Despite the existence of the above methods there is still a need for better iterative in vitro recombination methods for preparing novel polypeptide variants. Such methods should also be capable of being performed in small volumes, and amenable to automatisation.

### SUMMARY OF THE INVENTION

The present invention concerns briefly a method for producing recombined polynucleotides by utilizing necleotides or nucleotide analogues not normally present in naturally occurring polynucleotides, wherein the sugar-base bonds are cleavable, or from which the base-moiety can be cleaved, thus generating so-called AP-sites where the nucleotides or nucleotide analogues are present in the polynucleotide. These AP-sites may be used for generating random sized polynucleotide fragments for use in a shuffling procedure without the use of DNase I, or for blocking the polynucleotide synthesis at random positions in the polynucleotide, without the use of DNA adducts or DNA binding proteins or other such previously disclosed means.

More specifically, in a first aspect the present invention relates to a method for producing recombined polynucleotides, the method comprising the steps of:

i) providing a polynucleotide population comprising one or more nucleotide(s) or nucleotide analogue(s) different from dATP, dCTP, dGTP, and dTTP;

ii) excising the base-moiety of said nucleotide(s) or nucleotide-analogue(s) from the polynucleotide population of i) under conditions which promote cleavage of sugar-base bonds in polynucleotides, thereby generating one or more AP-site(s) in the polynucleotide population;

iii) annealing at least one primer to the polynucleotide population of ii) and extending the primer(s) by polynucleotide synthesis;

iv) dissociating the extended primer(s) of step iii) and the polynucleotide population, reannealing the extended primers to the polynucleotide population and further extending the primer(s) by polynucleotide syntesis; and optionally

v) repeating step iv) one or more times.

In a second aspect the invention also relates to a method for producing recombined polynucleotides, the method comprising the steps of:

i) providing a polynucleotide population comprising one or more nucleotide(s) or nucleotide analogue(s) different from dATP, dCTP, dGTP, and dTTP;

ii) excising the base-moiety of said nucleotide(s) or nucleotide-analogue(s) from the polynucleotide population of i) under conditions which promote cleavage of sugar-base bonds in polynucleotides, thereby generating one or more AP-site(s) in the polynucleotide population;

iii) cleaving the polynucleotide population of ii) at said AP-site(s);

iv) annealing at least one primer to the polynucleotide population of iii) and extending the primer(s) by polynucleotide synthesis;

v) dissociating the extended primer(s) of step iii) and the polynucleotide population, reannealing the extended primers to the polynucleotide population and further extending the primer(s) by polynucleotide syntesis; and optionally

vi) repeating step v) one or more times.

In a third aspect the invention relates to a method for producing recombined polynucleotides, the method comprising the steps of providing a polynucleotide population comprising one or more nucleotide(s) or nucleotide analogue(s) different from dATP, dCTP, dGTP, and dTTP, wherein said nucleotide(s) or nucleotide analogue(s) are suitable as targets for polynucleotide strand cleavage, cleaving said strands, and recombining and extending the products by polynucleotide synthesis.

In a fourth aspect the invention relates to a method for using recombined polynucleotides obtained by a method as defined in any of the previous aspects in identifying an encoded polypeptide having an activity of interest, where the polypeptide exhibits at least one altered property in comparison to known polypeptides that have the same activity, wherein said recombined polynucleotides are cloned into an appropriate vector, said vector is transformed into a suitable host cell wherein said encoded polypeptides are expressed, the polypeptides are screened in a suitable assay, an altered polypeptide of interest is identified, and the vector comprising the encoding polynucleotide is isolated.

In a final aspect the invention relates to a method for producing a polypeptide of interest as defined in the previous aspect, wherein the polynucleotide encoding the polypeptide of interest is cloned into a suitable expression vector and transformed into a suitable host cell which is cultivated under conditions suitable for expression of said polypeptide, and optionally the polypeptide is recovered.

Definitions

Prior to discussing this invention in further detail, the following terms will first be defined. The term “shuffling” means recombination of nucleotide sequence fragments of two or more homologous polynucleotides resulting in output polynucleotides (i.e. polynucleotides having been subjected to a shuffling cycle) having a number of nucleotide fragments exchanged, in comparison to the input polynucleotides (i.e. starting point homologous polynucleotides).

“Homology of DNA sequences or polynucleotides”: In the present context the degree of DNA sequence homology is determined as the degree of identity in percent between two sequences. The %-identity may suitably be determined by means of computer programs known in the art, such as GAP provided in the GCG program package (Program Manual for the Wisconsin Package, Version 8, August 1994, Genetics Computer Group, 575 Science Drive, Madison, Wis., USA 53711)(Needleman, S. B. and Wunsch, C. D., (1970), Journal of Molecular Biology, 48, 443-453). Using the computer program GAP (vide supra) with the following settings for DNA sequence comparison: GAP creation penalty of 5.0 and GAP extension penalty of 0.3.

“Primer”: The term “primer” used herein especially in connection with a polymerase chain reaction is an oligonucleotide (especially a “PCR-primer”) defined and constructed according to general standard specifications known in the art (“PCR A practical approach” IRL Press, (1991)).

“A primer directed to a sequence”: The term “a primer directed to a sequence” means that the primer (preferably to be used in a PCR reaction) is designed to exhibit at least 80% degree of sequence identity to the sequence fragment of interest, more preferably at least 90% degree of sequence identity to the sequence fragment of interest, which said primer consequently is “directed to”. The primer is designed to specifically anneal at the sequence fragment or region it is directed towards at a given temperature. Especially identity at the 3′ end of the primer is essential as is well known in the art.

“Random primer”: The primer to be used may be a completely random primer having a length of at least 6 nucleotides, such as: 5′-NNNNNN (N denotes that any of the four nucleotides A, T, G, or C is incorporated into the N-position during primer synthesis).

“Semi-random primer”: The primer comprises one or more regions that are random as well as one or more regions that are specific or are directed to a template sequence.

“Mutagenic primer”: A mutagenic primer is a specific primer in which one or more mismatches has been introduced into the DNA sequence at specific positions, thereby introducing mutations into the PCR-product at desired positions.

“Ramping”: The term “ramping” used herein especially in connection with a PCR reaction is to be understood as the transition phase between the annealing step in a PCR-cycle and the denaturation step, during which transition the temperature increases from the annealing temperature, typically between 10° C.-80° C., to the denaturation temperature, typically between 90° C.-100° C.

“AP-site”: An AP-site is an apurinic or apyrimidinic site which in the present context means a nulceotide or nucleotide analogue comprised in a DNA-strand, where the base-moiety of said nucleotide or nucleotide analogue has been removed by cleavage of the sugar base bond.

“Polypeptide”: Polymers of amino acids sometimes referred to as proteins. The sequence of amino acids determines the folded conformation that the polypeptide assumes, and this in turn determines biological properties and activity. Some polypeptides consist of a single polypeptide chain (monomeric), whereas other comprise several associated polypeptides (multimeric). All enzymes and antibodies are polypeptides.

“Enzyme”: A protein capable of catalysing chemical reactions. Specific types of enzymes to be mentioned are hydrolases, lyases, ligases, transferases, isomerases, and oxidoreductases.

The term “a gene” denotes herein a gene (a polynucleotide) which is capable of being expressed into a polypeptide within a living cell or by an appropriate expression system. Accordingly, said gene is defined as an open reading frame starting from a start codon (normally “ATG”, “GTG”, or “TTG”) and ending at a stop codon (normally “TAA”, TAG” or “TGA”). In order to express said gene there must be elements, as known in the art, in connection with the gene, necessary for expression of the gene within the cell. Such standard elements may include a promoter, a ribosomal binding site, a termination sequence, and maybe others elements as known in the art.

The term “substantially pure polynucleotide” as used herein refers to a polynucleotide preparation, wherein the polynucleotide has been removed from its natural genetic milieu, and is thus free of other extraneous or unwanted coding sequences and is in a form suitable for use within genetically engineered protein production systems.

Thus, a substantially pure polynucleotide contains at the most 10% by weight of other polynucleotide material with which it is natively associated (lower percentages of other polynucleotide material are preferred, e.g. at the most 8% by weight, at the most 6% by weight, at the most 5% by weight, at the most 4% at the most 3% by weight, at the most 2% by weight, at the most 1% by weight, and at the most ½% by weight). A substantially pure polynucleotide may, however, include naturally occurring 5′ and 3′ untranslated regions, such as promoters and terminators.

It is preferred that the substantially pure polynucleotide is at least 92% pure, i.e. that the polynucleotide constitutes at least 92% by weight of the total polynucleotide material present in the preparation, and higher percentages are preferred such as at least 94% pure, at least 95% pure, at least 96% pure, at least 96% pure, at least 97% pure, at least 98% pure, at least 99%, and at the most 99.5% pure.

The polynucleotides disclosed herein are preferably in a substantially pure form. In particular, it is preferred that the polynucleotides disclosed herein are in “essentially pure form”, i.e. that the polynucleotide preparation is essentially free of other polynucleoude material with which it is natively associated. Herein, the term “substantially pure polynucleotide” is synonymous with the terms “isolated polynucleotide” and “polynucleotide in isolated form”.

The term “denaturing” is used herein as known in the art, for example a double-stranded polynucleotide comprised in a liquid solution may be denatured by heating the solution to at least the melting-point or melting-temperature of the double-stranded polynucleotide and keeping the solution at that temperature until the double-stranded polynucleotide has denatured, separated, or “melted” into two complementary single-stranded polynucleotides.

“Annealing” as used herein means that conditions such as temperature and salt-concentrations in a liquid solution are so that a single-stranded polynucleotide comprised in the solution will anneal preferentially to another single-stranded homologous polynucleotide comprised in the solution, in other words polynucleotides that are not homologous will not anneal to any significant extent.

“Nucleic acid construct” when used herein, the term nucleic acid construct means a nucleic acid molecule, either single-or double-stranded, which is isolated from a naturally occurring source or which has been modified to contain segments of nucleic acids in a manner that would not otherwise exist in nature. The term nucleic acid construct is synonymous with the term expression cassette” when the nucleic acid construct contains the control sequences required for expression of a coding sequence of the present invention.

“Control sequence” is defined herein to comprise all components that are necessary or advantageous for the expression of a polynucleotide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

“Operably linked” is defined herein as a configuration in which a control sequence is appropriately placed at a position relative to the coding sequence of the polynucleotide sequence such that the control sequence directs the expression of the polynucleotide.

“Coding sequence” is intended to cover a polynucleotide sequence, which directly specifies the amino acid sequence of its protein product. The boundaries of the coding sequence are generally determined by an open reading frame, which usually begins with the ATG start codon. The coding sequence typically include DNA, cDNA, and recombinant nucleotide sequences.

In the present context, the term “expression” includes any step involved in the production of a polypeptide including, but not limited to, transcription, post-transcriptional modification, translation, post-translational modification, and secretion.

In the present context, the term “expression vector” covers a polynucleotide molecule, linear or circular, that comprises a polynucleotide segment encoding a polypeptide of interest, and which is operably linked to additional segments that provide for the expression.

In the present context, the term “allelic variant” denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

The term “thermostable” protein(s) in the present context means that the protein(s) remains essentially functional after having been exposed to the relatively high temperatures needed to denature the double-stranded polynucleotides in step (b) of the method of the invention. Specifically the thermostable protein(s) retains from at least 60% to 80% of its activity at its optimum temperature after one denaturing step; wherein the activity may be determined by the ATP-hydrolysis (ATPase) assay described in (Biswas and Hsieh, 1996, vide supra) which is incorporated herein by reference.

The techniques used to isolate or clone a polynucleotide sequence are known in the art and include isolation from genomic DNA, preparation from cDNA, or a combination thereof. The cloning of the polynucleotide sequences of the present invention from such genomic DNA can be effected, e.g., by using the well known polymerase chain reaction (PCR), expression cloning, or antibody screening of expression libraries to detect cloned DNA fragments with shared structural features. See, e.g., Innis et al., 1990, PCR: A Guide to Methods and Application, Academic Press, New York. Other amplification procedures such as ligase chain reaction (LCR), ligated activated transcription (LAT) and nucleotide sequence-based amplification (NASBA) may be used. The nucleotide sequence may be cloned from a bacterial or fungal strain or another or related organism and thus, for example, may be an allelic or species variant of the polypeptide encoding region of the nucleotide sequence.

The polynucleotide sequence may be obtained by standard cloning procedures used in genetic engineering to relocate the polynucleotide sequence from its natural location to a different site where it will be reproduced. The cloning procedures may involve excision and isolation of a desired polynucleotide fragment comprising the polynucleotide sequence of interest, insertion of the fragment into a vector molecule, and incorporation of the resulting recombinant vector into a host cell where multiple copies or clones of the polynucleotide sequence will be replicated. The polynucleotide sequence may be of genomic, cDNA, RNA, semi synthetic, synthetic origin, or any combinations thereof.

There is a substantial commercial interest in polypeptides such as pharmaceutically active peptides or industrial enzymes, and there is much research focused on changing or improving the properties or activities of such polypeptides. Terms like “protein engineering” or “gene shuffling” are frequently encountered in the art. The present invention provides a new way of recombining polynucleotide sequences without having to fragment the template polynucleotides or synthesize a large number of overlapping primers to be used in a PCR reaction etc.

It is well known in the art that polynucleotide sequences encoding certain polypeptides with similar properties or activities, such as enzymes, are often highly homologous. The homologous polynucleotides and polypeptides may be species variants or allelic variants descending from a common ancestral sequence which have evolved separately to the present day.

A template polynucleotide may encode an enzymatic polypeptide e.g. an aminopeptidase, an amylase, a carbohydrase, a carboxypeptidase, a catalase, a cellulase, a chitinase, a cutinase, a cyclodextrin glycosyltransferase, a deoxyribonuclease, an esterase, an alpha-galactosidase, a beta-galactosidase, a glucoamylase, an alpha-glucosidase, a beta-glucosidase, a haloperoxidase, an invertase, a laccase, a lipase, a mannosidase, an oxidase, a pectinolytic enzyme, a peroxidase, a phytase, a polyphenoloxidase, a proteolytic enzyme, a ribonuclease, or a xylanase.

The present invention also relates to nucleic acid constructs comprising a nucleotide sequence of the present invention operably linked to one or more control sequences that direct the expression of the coding sequence in a suitable host cell under conditions compatible with the control sequences.

A polynucleotide sequence of the present invention may be manipulated in a variety of ways to provide e.g. for expression of an encoded polypeptide. Manipulation of the nucleotide sequence prior to its insertion into a vector may be desirable or necessary depending on the expression vector. The techniques for modifying nucleotide sequences utilizing recombinant DNA methods are well known in the art.

The control sequence may be an appropriate promoter sequence, a nucleotide sequence which is recognized by a host cell for expression of the nucleotide sequence. The promoter sequence contains transcriptional control sequences, which mediate the expression of the polypeptide. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al, 1978, Proceedings of the National Academy of Sciences USA 75: 3727-3731), as well as the tac promoter (DeBoer et al, 1983, Proceedings of the National Academy of Sciences USA 80: 21-25). Further promoters are described in “Useful proteins from recombinant bacteria” in Scientific American, 1980, 242: 74-94; and in Sambrook et al, 1989, supra.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA which is important for translation by the host cell. The leader sequence is operably linked to the 5′ terminus of the nucleotide sequence encoding the polypeptide. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

The control sequence may also be a signal peptide coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding regions for filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

Useful signal peptides for yeast host cells are obtained from the genes for Saccharomyces cerevisiae alpha-factor and Saccharomyces cerevisiae invertase. Other useful signal peptide coding regions are described by Romanos et al., 1992, supra.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtillis neutral protease (nprT), Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which Is amplified in the presence of methotrexate, and the metallothionein genes which are amplified with heavy metals. In these cases, the nucleotide sequence encoding the polypeptide would be operably linked with the regulatory sequence.

The present invention also relates to recombinant expression vectors comprising the polynucleotides of the invention especially when those are comprised in a nucleic acid construct such as an expression vector. The various nucleotide and control sequences described above may be joined together to produce a recombinant expression vector which may include one or more convenient restriction sites to allow for insertion or substitution of the polynucleotide sequence at such sites.

Alternatively, a polynucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about the expression of the nucleotide sequence. The choice of the vector will typically depend on the compatibility of the vector with the host cell into which the vector is to be introduced. The vectors may be linear or closed circular plasmids.

The vector may be an autonomously replicating vector, ie., a vector which exists as an extrachromosomal entity, the replication of which is independent of chromosomal replication, e.g., a plasmid, an extrachromosomal element, a minichromosome, or an artificial chromosome.

The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

Examples of bacterial selectable markers are the daI genes from Bacillus subtilis or Bacillus licheniformis, or markers which confer antibiotic resistance such as ampicillin, kanamycin, chloramphenicol or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof.

Preferred for use in an Aspergillus cell are the amdS and pyrG genes of Aspergillus nidulans or Aspergillus oryzae and the bar gene of Streptomyces hygroscopicus.

The vectors of the present invention preferably contain an element(s) that permits stable integration of the vector into the host cell's genome or autonomous replication of the vector in the cell independent of the genome.

For integration into the host cell genome, the vector may rely on the nucleotide sequence encoding the polypeptide or any other element of the vector for stable integration of the vector into the genome by homologous or nonhomologous recombination.

Alternatively, the vector may contain additional nucleotide sequences for directing integration by homologous recombination into the genome of the host cell. The additional nucleotide sequences enable the vector to be integrated into the host cell genome at a precise location(s) in the chromosome(s).

To increase the likelihood of integration at a precise location, the integrational elements should preferably contain a sufficient number of nucleotides, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus. Examples of origins of replication for use in a yeast host cell are the 2 micron origin of replication, ARS1, ARS4, the combination of ARS1 and CEN3, and the combination of ARS4 and CEN6. An example of a filamentous fungal stabilizing element is the AMA1 sequence. The origin of replication may be one having a mutation which makes its functioning temperature-sensitive in the host cell (see, e.g., Ehrlich, 1978, Proceedings of the National Academy of Sciences USA 75: 1433).

More than one copy of a nucleotide sequence of the present invention may be inserted into the host cell to increase production of the gene product. An increase in the copy number of the nucleotide sequence can be obtained by integrating at least one additional copy of the sequence into the host cell genome or by including an amplifiable selectable marker gene with the nucleotide sequence where cells containing amplified copies of the selectable marker gene, and thereby additional copies of the nucleotide sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

The present invention also relates to recombinant a host cell comprising the polynucleotide(s) or nucleic acid construct(s) of the invention, which are advantageously used in the screening assays described herein. A vector comprising a nucleotide sequence of the present invention is introduced into a host cell so that the vector is maintained as a chromosomal integrant or as a self-replicating extra-chromosomal vector as described earlier.

The host cell may be a unicellular microorganism, e.g., a prokaryote, or a non-unicellular microorganism, e.g., a eukaryote.

Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp.

In a preferred embodiment, the bacterial host cell is a Bacillus lentus, Bacillus licheniformis, Bacillus stearothermophilus, or Bacillus subtilis cell. In another preferred embodiment, the Bacillus cell is an alkalophilic Bacillus.

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

The host cell may be a eukaryote, such as a mammalian, insect, plant, or fungal cell.

In a preferred embodiment, the host cell Is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In an even more preferred embodiment, the yeast host cell is a Candida, Hansenula, Kluyveromyces, Pichia, Saccharomyces, Schizosaccharomyces, or Yarrowia cell.

In a most preferred embodiment, the yeast host cell is a Saccharomyces carisbergensis, Saccharomyces cerevisiae, Saccharomyces diastaticus, Saccharomyces douglasii, Saccharomyces kluyveri, Saccharomyces norbensis or Saccharomyces oviformis cell. In another most preferred embodiment, the yeast host cell is a Kluyveromyces lactis cell. In another most preferred embodiment, the yeast host cell is a Yarrowia lipolytica cell.

In another more preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, or Trichoderma.

In a most preferred embodiment, the filamentous fungal host cell is an Aspergillus awamori, Asperillus foetidus, Aspergillus japonicus, Aspergillus nidulans, Aspergillus niger or Aspergillus oryzae cell. In another most preferred embodiment, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In an even most preferred embodiment, the filamentous fungal parent cell is a Fusarium venenatum (Nirenberg sp. nov.) cell. In another most preferred embodiment, the filamentous fungal host cell is a Humicola insolens, Humicola lanuginosa, Mucor miehei, Myceliophthora thermophila, Neurospora crassa, Penicillium purpurogenum, Thielavia terrestris, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### DETAILED DESCRIPTION OF THE INVENTION

The present invention relates to methods for producing recombined polynucleotides without in one aspect without fragmenting the starting polynucleotides and in another without the use of DNase I, and generally without the use of DNA adducts.

The method according to the present invention relies on the activity of several enzymes termed DNA glycosylases, which catalyze the cleavage of base-sugar bonds in DNA. These DNA glycosylases have the common property of acting only on altered or damaged nucleotide residues in DNA. Double-stranded DNA is the preferred substrate for all the known DNA glycosylases except for uracil-DNA glycosylase, which is the only known glycosylase, which also acts on single-stranded DNA. Removal of the base-moiety from the nucleotide by the DNA glycosylase leads to the formation of an apurinic or apyrimidinic site, herein termed “AP-site”. When a substrate DNA template, containing one or more AP-site(s), is used in an amplification protocol such as a polymerase chain reaction (PCR) or in a primer extension, the DNA-polymerase stalls at the AP-site and polynucleotide extension stops.

A template polynucleotide containing one or more AP-site(s) in unknown positions will during an amplification reaction such as a PCR or a primer extension, using a plurality of specific, semi-random, or random primers, give rise to the formation of a population of randomly sized polynucleotide fragments, which may then be recombined or “shuffled” resulting in a population of recombined polynucleotides, that are homologous to the starting polynucleotide population.

The said polynucleotide(s) comprising one or more AP-sites can be constructed from a starting polynucleotide population by incorporating any nucleotide or nucleotide analogue, which can be recognized and cleaved by a suitable DNA-glycosylase, releasing the base-moiety.

One aspect of the present invention relates to a method for producing recombined polynucleotides, the method comprising the steps of: i) providing a polynucleotide population comprising one or more nucleotide(s) or nucleotide analogue(s) different from dATP, dCTP, dGTP, and dTTP; ii) excising the base-moiety of said nucleotide(s) or nucleotide-analogue(s) from the polynucleotide population of i) under conditions which promote cleavage of sugar-base bonds in polynucleotides, thereby generating one or more AP-site(s) in the polynucleotide population; iii) annealing at least one primer to the polynucleotide population of ii) and extending the primer(s) by polynucleotide synthesis; iv) dissociating the extended primer(s) of step iii) and the polynucleotide population, reannealing the extended primers to the polynucleotide population and further extending the primer(s) by polynucleotide syntesis; and optionally v) repeating step iv) one or more times.

Another aspect of the invention relates to a method for producing recombined polynucleotides, the method comprising the steps of: i) providing a polynucleotide population comprising one or more nucleotide(s) or nucleotide analogue(s) different from dATP, dCTP, dGTP, and dTTP; ii) excising the base-moiety of said nucleotide(s) or nucleotide-analogue(s) from the polynucleotide population of i) under conditions which promote cleavage of sugar-base bonds in polynucleotides, thereby generating one or more AP-site(s) in the polynucleotide population; iii) cleaving the polynucleotide population of ii) at said AP-site(s); iv) annealing at least one primer on the polynucleotide population of iii) and extending the primer(s) by polynucleotide synthesis; v) dissociating the extended primer(s) of step iii) and the polynucleotide population, reannealing the extended primers to the polynucleotide population and further extending the primer(s) by polynucleotide syntesis; and optionally vi) repeating step v) one or more times.

For providing e.g. DNA polynucleotides comprising uracil, the starting polynucleotide is mixed with an appropriate DNA polymerase, dATP, dCTP, dGTP, dTTP, and dUTP, a suitable buffer and a pair of primers that will allow amplification of the region of interest. The said DNA polymerase comprises in one embodiment a thermostable DNA polymerase such as Taq-polymerase, Amplitaq®-polymerase, Vent®-polymerase, Pwo-polymerase, Pfu-polymerase, Tth-polymerase or mixtures thereof.

In another embodiment of the invention the DNA polymerase may be added after each PCR-cycle, if the polymerase is not thermostable, such as T4 polymerase, T7 polymerase, E. coli DNA-polymerase I, the Klenow fragment of DNA-polymerase I. The concentration of dUTP and dTTP in the reaction can be varied to obtain different incorporation ratios between dUTP and dTTP. Normally the concentration of dTTP is between 10 μM and 350 μM, and the concentration of dUTP is between 10 μM and 350 μM. An example of a reaction is with a dUTP concentration at 40 μM and dTTP at 210 μM. Another example is with a dUTP concentration at 100 μM and dTTP at 150 μM. The concentration of dATP, dCTP, and dGTP can be varied but is normally around 100-300 μM.

The mixture is placed in a PCR thermocycler in a suitable tube. The thermocycler is heated to a temperature of 90-100° C. for a period of time (typically 1-10 min) in order to denature the DNA templates (typically 90-100° C. for 0-5 minutes). Then the temperature is lowered (typically between 10° C. and 90° C. for 0-5 minutes) to allow annealing of the primer to the single-stranded template. The temperature is then raised to allow extension of the primers along the template (typically 5-180 seconds at 66-76° C.). After extension the temperature is raised to 96° C. thereby denaturing the extended primers and the template. This cycle of denaturation, annealing, and extension can be repeated, typically between 1 and 99 times. The generated uracil containing PCR product is subsequently purified either on an agarose gel, by beads (using an affinity label on either templates or primers), or through columns. In cases where the template DNA used in the above PCR reaction is methylated by the Dam methyl transferase, it is convenient to add the restriction endonuclease DpnI to select against parental DNA. DpnI recognises the target sequence 5′-Gm6ATC-3′, where the adenine residue is methylated. DNA isolated from most common strains of E. coli is methylated at GATC-sites. In one embodiment according to the invention the product of step i) is treated with a restriction endonuclease such as DpnI, before performing step ii).

The purified uracil containing DNA is then mixed in a suitable tube with the appropriate buffer and the enzyme uracil-DNA-glycosylase (UDG), normally using excess UDG based on the calculation that 1 unit of UDG will release all the base moieties of all uracil-bases from 1 μg single-stranded uracil containing DNA, at 37° C. in 60 minutes. Typically the uracil-containing DNA is incubated with UDG for 1-24 hours, whereby the DNA is deuracilated.

In a first aspect of the invention the deuracilated, but not piperidine treated DNA, encoding e.g. different enzyme variants of the same gene or different enzymes having the same type of activity encoded by homologous genes, is then mixed in a suitable tube together with a DNA polymerase, dNTP's, a suitable buffer, and primers (being either random oligomers of 6-30 nucleotides, specific oligomers of 6-50 nucleotides, or mutagenic oligomers of 6-30 nucleotides, or a combination thereof). The mixture is placed in a thermo cycler in a suitable tube and the below cycles are performed one or more times:

The template is denatured (typically 90-100° C. for 0-5 minutes). Then the temperature is lowered to allow annealing of the primers to the single-stranded templates (typically to a value between 10° C. and 90° C. for 0-5 minutes). Now the temperature is raised again to the denaturation temperature (90-100° C.) allowing some extension of the primer to be synthesised by the DNA polymerase during ramping. Alternatively a short extension period (typically 0-30 seconds at 70-75° C.) can be introduced to allow larger extensions of the primers to be generated. When the extension products reach a deuracilated site on the templates the polymerase stalls and extension stops. Thereafter the temperature is increased to 96° C. for 15 seconds, whereby denaturation takes place and the extended primers and templates are separated. The temperature is then lowered to annealing temperature, whereby extended products and primers can re-anneal to the DNA templates or to other extended products at regions of shared homology. This re-annealing will occur in a recombinative manner such that a primer extended on e.g. variant A in the first cycle, will anneal to e.g. template DNA from variant B In the second cycle, whereby crossover between different enzyme variants or homologous enzymes will be generated.

As this above procedure can be repeated (typically between 1 to 99 cycles), large numbers of different crossover events will occur and a vast number of different molecules will be generated.

Having performed the desired number of cycles the generated recombined DNA polymers can be purified from the oligomers used as primers. One way is to isolate and clone a specific amplified band containing the gene coding for the polypetide of interest into a suitable vector. This can be done either on an agarose gel (typically used for isolating fragments between 50 to 1000 base pairs), by affinity beads (using an affinity label on either templates or primers), or through columns.

In a second aspect according to the present invention the deuracilated DNA described above is precipitated from solution by one of the methods well known to those skilled in the art, e.g. by addition of sodium acetate and ethanol. The precipitated DNA is washed and dried and then dissolved in an adequate amount of 1M piperidine (typically between 10-1000 μl) and placed on a heating block at 90° C. for 20 min. Thereafter the tube is placed in a vacuum desiccator and the piperidine is evaporated until the tube is dry. The DNA, which is now fragmented, is dissolved in water or a suitable buffer. As an alternative to piperidine treatment it is possible to use specific endonucleases that cleaves the DNA at the AP-sites. Such endonucleases comprises the E. coli endonuclease IV, a class II AP-endonuclease that cleaves at apyrimidinic sites and has no associated exonuclease activity.

The DNA fragments generated after treatment of the deuracilated DNA (encoding e.g. different enzyme variants of the same gene or different enzymes having the same type of activity encoded by homologous genes) with piperidine are mixed with a DNA polymerase, dNTP's, and a suitable buffer, and then placed in a PCR thermo cycler.

The thermo cycler is heated to a temperature of 96° C. for 2 minutes in order to denature the DNA templates. Thereafter the following cycle is performed: Denaturation of templates at 96° C. for 15 seconds. Lowering of the temperature to a value between 10° C. and 70° C. for 20 seconds to allow annealing of the fragmented DNA to complementary strands. Raising the temperature to 96° C. for 30 seconds. During the ramping period the polymerase will extend annealed DNA fragments from the 3′-ends. As the temperature increases during the ramping, denaturation takes place and the extended fragments are separated. The temperature is thereafter lowered to annealing temperature thereby allowing extended products to re-anneal. This re-annealing will occur in a recombinative manner such that a DNA fragment extended on e.g. variant A in the first cycle, will anneal to e.g. template DNA from variant B in the second cycle, whereby crossover between different enzyme variants or homologous enzymes will be generated. As cycle of denaturing, re-annealing, and extension can be repeated (typically between 1 to 99 cycles) large numbers of different crossover events will occur and a vast number of different molecules will be generated.

The generated recombined library of DNA polymers as illustrated in the two S alternative aspects of the present invention can subsequently be amplified In a standard PCR reaction (e.g. 94° C., 5 min; 25 cycles of (94° C., 30 sec; 55° C., 30 sec; 72° C., 2 min); 72° C., 5 min; 4° C.). The final PCR amplification can also introduce specific restriction endonuclease recognition sites to facilitate cloning of the population of recombined polynucleotides.

After cloning of the recombined libraries of DNA polymers, produced by the methods according to the invention, into a suitable vector, the libraries can be expressed in a suitable host organism using standard expression vectors and corresponding expression systems known in the art.

A preferred embodiment of the invention relates to a method of the first or second aspects, wherein the polynucleotide population of step i) or the primer extending is provided by performing a polymerase chain reaction with at least one DNA polymerase or with a mixture of at least two DNA polymerases, preferably with one or more DNA polymerase(s) chosen from the group consisting of: Taq-polymerase, Amplitaq®-polymerase, Vent®-polymerase, Pwo-polymerase, Pfu-polymerase, Tth-polymerase, T4 polymerase, T7 polymerase, E. coli DNA-polymerase I, Stoffel fragment, and Klenow fragment of DNA-polymerase I.

The arrest of the polymerase reaction may be obtained in different ways, such as by raising the temperature, or adding specific reagents as described in WO 95/17413. When raising the temperature for this purpose, it is preferred to use temperatures between 90° C. and 99° C. It is also possible to use chemical agents e.g. DMSO, procedures are mentioned in e.g. WO 95/17413.

Another preferred embodiment relates to a method of the first or second aspects, wherein the polynucleotide population of step i) is isolated from a host cell which is capable of incorporating nucleotide(s) or nucleotide analogue(s) different from dATP, dCTP, dGTP, and dTTP into a polynucleotide during polynucleotide replication or in vivo synthesis.

Yet another preferred embodiment relates to a method of the first or second aspects, wherein the polynucleotide population of step i) is provided by chemical synthesis.

Regarding the primers used in all aspects of the present invention, preferred embodiments relate to a method, wherein said primer(s) comprises one or more random or semi-random primers; or wherein said primer(s) comprises one or more mutagenic primers, or even wherein said primer(s) comprises one or more specific primers.

The incorporation of uracil, as dUTP, or uracil analogues such as 5-fluorouracil, as 5-fluoro-dUTP, into DNA and the subsequent excision of the incorporated uracil-base moieties from the DNA by use of the enzyme uracil-DNA glycosylase is one example of a suitable nucleotide or nucleotide analogue and a corresponding DNA glycosylase.

A preferred embodiment relates to a method of all aspects, wherein said nucleotide(s) or nucleotide analogue(s) comprises dUTP, 5fluoro-dUTP, dITP, 3-methyl-dATP, 7-methyl-dATP, 7-methyl-dGTP, or a mixture of these.

One preferred embodiment relates to a method of all aspects, wherein the rate, in the polynucleotide population of step i) of each nucleotide or nucleotide analogue that is different from dATP, dCTP, dGTP, and dTTP to the corresponding naturally occurring nucleotide(s), is controlled by optimizing the ratio of said nucleotide(s) or nucleotide analogue(s) to the corresponding naturally occurring nucleotide(s) during synthesis of the polynucleotide population of step i). In a preferred embodiment the nucleotide dUTP is used and the dUTP/dTTP ratio is about 0.02-1.5, more preferably the dUTP/dTTP ratio is about 0.1-0.8.

The polynucleotide population comprising one or more nucleotides or nucleotide analogues different from dATP, dCTP, dGTP, and dTTP is subsequently treated with an enzyme, such as a DNA glycosylase, which specifically recognises and cleaves the sugar-base bond in the said nucleotide or nucleotide analogue. The choice of DNA glycosylase depends on which nucleotide or nucleotide analogue is used.

A preferred embodiment relates to a method of all aspects, wherein the DNA-glycosylase is an uracil-DNA glycosylase, a hypoxanthine-DNA glycosylase, a 3-methyladenine-DNA glycosylase I, a 3-methyladenine-DNA glycosylase II, a formamidopyrimidine-DNA glycosylase, or a mixture of these.

Other combinations of nucleotide analogues and DNA glycosylases are dITP/hypoxanthine-DNA glycosylase, 3-methyl-dATP/3-methyladenine-DNA glycosylase 1,3-methyl-dATP/3-methyladenine-DNA glycosylase II, 3-methyl-dGTP/3-methyladenine-DNA glycosylase II, 7-methyl-dATP/3-methyladenine-DNA glycosylase II, 7-methyl-dGTP/3-methyladenine-DNA glycosylase II, and 7-methyl-dGTP/formamidopyrimidine-DNA glycosylase.

The method of the invention uses annealing of primers to the templates. In this context said annealing may be random or specific, meaning either anywhere on the polynucleotide or at a specific position depending on the nature of the primer.

In providing a polynucleotide population comprising one or more nucleotides or nucleotide analogues different from dATP, dCTP, dGTP, and dTTP the said primers are preferably specific, however, random or semi-random primers, or mutagenic primers might also work.

For providing random polynucleotide fragments by annealing and extension of primers on polynucleotides comprising AP-sites, the said primers are random, semi-random, specific, or mutagenic, or a mixture thereof.

If the extended primers produced are to be separated from the primers during the process it is convenient to use labeled templates in order to provide a simple means for separation. A preferred label is biotin or digoxigenin.

After cleavage of the sugar-base bond and removal of the base moiety, primers are annealed and extended at least once, on the product of step ii) above. The said primers comprise a population of random primers, semi-random primers, specific primers, or mutagenic primers, and in a specific embodiment annealing and extension is done by a) denaturing the polynucleotide population of ii) containing AP-sites to produce single-stranded templates; b) annealing said primers to the single-stranded templates; c) extending said primers by initiating DNA synthesis by the use of said primers, dATP, dCTP, dGTP, dTTP, and a DNA-polymerase.

Another way to generate random fragments after providing a population of polynucleotides comprising one or more nucleotides or nucleotide analogues different from dATP, dCTP, dGTP, and dTTP, according to step i) above and subsequent cleavage of the sugar-base bonds according to step ii) above, is to cleave the product(s) of ii) at the AP-sites, thereby generating random polynucleotide fragments, and subsequently recombine and extend the said random fragments generated by said cleavage at the AP-sites. wherein the cleaving at the AP-site(s) is done by using one or more AP-endonuclease, preferably an AP-endonuclease chosen from the group consisting of Escherichia coli exonuclease III, E. coli endonuclease IV, and E. coli endonuclease V; or a mammalian AP endonuclease.

A preferred embodiment relates to a method of the second aspect, wherein the cleaving at the AP-site(s) is done by using one or more AP-endonuclease(s), preferably an AP-endonuclease chosen from the group consisting of Escherichia coli exonuclease III, E. coli endonuclease IV, and E. coli endonuclease V; or a mammalian AP endonuclease; or a mixture of these.

Another preferred embodiment relates to a method of the second aspect, wherein the cleaving at the AP-site(s) is done by using piperidine as exemplified herein in a non-limiting example.

One more preferred embodiment relates to a method of the second aspect, wherein the cleaving at the AP-site(s) is done by increasing the temperature to more than 50° C., or 60° C., or 70° C., or even more than 80° C., and/or alkaline conditions, preferably with a pH of at least 8, more preferably at least 9, even more preferably at least 10, and most preferably at least 11.

In the method of the invention the starting polynucleotide population may be provided as PCR-fragments, plasmid DNA, phage DNA, phagemid DNA, or genomic DNA. The starting polynucleotide population may originate from wild type organisms of different genera or species or even different strains of same species, it may comprise mutant variants of the same native polynucleotide, or it may comprise homologous polynucleotides isolated from nature, or combinations of these.

It may be advantageous to use pre-selected polynucleotide populations in the method of the invention, the polynucleotides comprising mutations resulting in one or more altered or improved property(ies) of interest. The present method of the invention may then recombine said polynucleotides for subsequent screening for one or more even further altered and/or improved property(ies) of interest. Such pre-selected populations may be identified by standard procedures in the art comprising e.g. error-prone PCR of templates of interest followed by screening/selection for templates with the characteristics of interest. The mutagenesis frequency (low or high mutagenesis frequency) of the error-prone PCR step is preferably adjusted in relation to the subsequent screening capacity, i.e. if the screening capacity is limited the error-prone PCR frequency is preferably low (i.e. one to two mutations in each template) (see WO 92/18645 for further details).

A preferred embodiment relates to a method according to all aspects, wherein the polynucleotide population of step i) comprises mutants or variants of the same native polynucleotide, or comprises homologous polynucleotides isolated from nature, or both.

Another preferred embodiment relates to a method according to all aspects, wherein at least one individual polynucleotide of the population of step i) exhibits a nucleotide sequence %-identity of at least 50%, preferably 60%, more preferably 70%, still more preferably 80%, even more preferably 90%, or most preferably at least 95% to at least one other polynucleotide of the population.

Still another preferred embodiment relates to a method according to all aspects, wherein the polynucleotide population of step i) originates from at least two wild type organisms of different genera or preferably from different species.

Yet another preferred embodiment relates to a method according to all aspects, wherein said the polynucleotide population of step i) is cloned into a suitable vector, preferably the vector is a plasmid.

In a preferred embodiment the polynucleotide population of step I) comprises polynucleotides encoding at least one enzyme, preferably at least a hydrolase, a lyase, a ligase, a transferase, an isomerase, or an oxidoreductase.

In another preferred embodiment the polynucleotide population of step i) comprises polynucleotides encoding at least one polypeptide or peptide having antimicrobial activity.

In still another preferred embodiment the polynucleotide population of step i) comprises at least one polynucleotide encoding a polypeptide having biological activity; preferably the polypeptide is insulin, pro-insulin, pre-pro-insulin, glucagon, somatostatin, somatotropin, thymosin, parathyroid hormone, pituitary hormones, somatomedin, erythro-poietin, luteinizing hormone, chorionic gonadotropin, hypothalamic releasing factor, antidiuretic hormone, blood coagulant factor, thyroid stimulating hormone, relaxin, interferon, thrombopoeitin (TPO) or prolactin.

Also in a preferred embodiment the polynucleotide population of step i) comprises at least one polynucleotide which has a biological function, preferably in transcription initiation or termination, translational initiation, or as an operator site related to expression of one or more gene(s).

A number of suitable screening or selection systems to screen or select for a desired biological activity are described in the art. Examples are:

Strauberg et al. (Biotechnology 13: 669-673 (1995) describes a screening system for subtilisin variants having Calcium-independent stability; Bryan et al. (Proteins 1:326-334 (1986)) describes a screening assay for proteases having an enhanced thermal stability; and PCT-DK96/00322 describes a screening assay for lipases having improved wash performance in washing detergents.

If, for instance, the polypeptide in question is an enzyme and the desired improved functional property is the wash performance, the screening may conveniently be performed by use of a filter assay based on the following principle:

The recombination host cell is incubated on a suitable medium and under suitable conditions for the enzyme to be secreted, the medium being provided with a double filter comprising a first protein-binding filter and on top of that a second filter exhibiting a low protein binding capability. The recombination host cell is located on the second filter. Subsequent to the incubation, the first filter comprising the enzyme secreted from the recombination host cell is separated from the second filter comprising said cells. The first filter is subjected to screening for the desired enzymatic activity and the corresponding microbial colonies present on the second filter are identified.

The filter used for binding the enzymatic activity may be any protein binding filter e.g. nylon or nitrocellulose. The topfilter carrying the colonies of the expression organism may be any filter that has no or low affinity for binding proteins e.g. cellulose acetate or Durapore®. The filter may be pre-treated with any of the conditions to be used for screening or may be treated during the detection of enzymatic activity. The enzymatic activity may be detected by a dye, fluorescence, precipitation, pH indicator, IR-absorbance or any other 5 known technique for detection of enzymatic activity. The detecting compound may be immobilized by any immobilizing agent e.g. agarose, agar, gelatin, polyacrylamide, starch, filter paper, cloth; or any combination of immobilizing agents.

If the improved functional property of the polypeptide is not sufficiently good after one cycle of shuffling, the polypeptide may be subjected to another cycle.

Further aspects of the invention therefore relates to a method for using recombined polynucleotides obtained by a method as defined in any the previous aspects In Identifying an encoded polypeptide having an activity of interest, where the polypeptide exhibits at least one altered property in comparison to known polypeptides that have the same activity, wherein said recombined polynucleotides are cloned into an appropriate vector, said vector is transformed into a suitable host cell wherein said encoded polypeptides are expressed, the polypeptides are screened In a suitable assay, an altered polypeptide of interest is identified, and the vector comprising the encoding polynucleotide is isolated.

In a still further aspect the present invention relates to a method for producing a polypeptide of interest as defined in the previous aspect, wherein the polynucleotide encoding the polypeptide of interest is cloned into a suitable expression vector and transformed into a suitable host cell which is cultivated under conditions suitable for expression of said polypeptide, and optionally the polypeptide is recovered.

In the following the invention shall be further illustrated by some none limiting examples.

### EXAMPLE 1

### Construction of a Diversified Library of Laccase Variants by Assembly of Degraded DNA in the Presence of Mutagenic Oligonucleotides

A genomic fragment of the laccase from Coprinus cinereus was inserted into the A. oryzae expression vector pENI2149, to create plasmid pCC2. 20 ng of this plasmid was used as template for PCR in a total volume of 100 μl using 1 μM each of primers:

| | |
|---|---|
| SEQ ID NO: 1 | 5′-agggatgccatgcttggagtttcc |
| and | |
| SEQ ID NO: 2. | 5′-ccaattgccctcatccccatcc |

PCR was performed using 0.5 units Amplitaq® DNA polymerase, suppliers buffer, 250 μM each of dATP, dCTP and dGTP, 200 μM of dTTP, and 50 μM of dUTP.

PCR cycling was as follows: 94° C., 2 min; 25 cycles of (94° C., 30 sec; 55° C., 30 sec; 72° C., 2 min); 72° C., 5 minutes; 4° C. hold. Following the PCR reaction DNA was purified on a PCR-purification column (Qiagen®), and eluted into 100 μl 10 mM Tris-HCl, pH 7.5. 20 μl (20 units) UDG (NEB), 14 μl NE buffer 4, and 6 μl DpnI (NEB) were added, and the tube was incubated at 37° C. for 16 hrs. Thereafter DNA was precipitated by addition of 1/10 vol 3M NaAc, 2.5 vol 96% EtOH. Precipitated DNA was washed by 70% EtOH, dried and dissolved in freshly prepared IM piperidine. The tube was placed at 90° C. for 20 min and thereafter transferred to a vacuum desiccator to evaporate the piperidine solution. Dried DNA was dissolved in 50 μl 10 mM Tris-HCl and used for an assembly reaction together with the mutagenic primers La2-La11:

| | | |
|---|---|---|
| SEQ ID NO: 3 | La2: | 5′-ccgatctctccaggccaagctttcctc |
| | | tac |
| SEQ ID NO: 4 | La3: | 5′-gtagaggaaagcgcggcctggagagat |
| | | cgg |
| SEQ ID NO: 5 | La4: | 5′-acaatgaccctcaagctgccctctacg |
| SEQ ID NO: 6 | La5: | 5′-acaatgacccacgtgctgccctctacg |
| SEQ ID NO: 7 | La6: | 5′-gggagcggggatctggtaccaatcgg |
| SEQ ID NO: 8 | La7: | 5′-ggagggagcggggatgcgataccaatc |
| | | ggcgag |
| SEQ ID NO: 9 | La8: | 5′-ttactgagcctcaaacggttgatcgtc |
| | | tc |
| SEQ ID NO: 10 | La9: | 5′-ttactgagccgcgcacggttgatcgtc |
| | | tc |
| SEQ ID NO: 11 | La10: | 5′-ggtcgatgagagcctgcaggtcggctt |
| SEQ ID NO: 12 | La11: | 5′-ggtcgatgagagcgcggaggtcggctt |

An assembly reaction was performed as follows:

1.2 μg fragmented DNA

0.05 pmole of La2-La11

2.5 μl 10× Pwo-buffer

5 μl of a 2.5 mM dNTP solution

0.5 units of Pwo polymerase (Boehringer®)

H₂O to 25 μl

Cycling (Assembly1) was as follows: 94° C., 2min; (94° C., 30 sec; 40° C., 30 sec; 72° C., 45 sec)×25cycles; 4° C. hold.

5 μl of assembly1 was thereafter subjected to a new round of assembly PCR as follows:

5 μl assembly1

2.5 μl Pwo-buffer

2.5 μl of a 2,5 mM dNTP

0.5 units Pwo polymerase

H₂O to 25 μl

Cycling (Assembly2) was as follows: 94° C., 2min; (94° C., 30 sec; 40° C., 30 sec; 72° C., 45 sec)×25cycles; 4° C. hold.

A smear of DNA was seen between 600 and 4000 bp when assembly2 was analyzed by agarose gel electrophoresis. Half of assembly2 was then mixed in a tube with two specific primers as follows:

5 μl Pwo-buffer

5 μl of a 2.5mM dNTP solution

1 μl of a 100 μM solution of a forward primer (BamHI-fwd)

1 μl of a 100 μM solution of a reverse primer (2801-rev)

0.5 units Pwo polymerase

H2O to 50 μl

The primers were:

SEQ ID NO: 13; BamHI-fwd: 5′-cgtggatccttcaccatgttcaagaacctcctctcg

SEQ ID NO: 14; 2801-rev: 5′-ggattgattgtctaccgccag

Cycling was as follows: 94° C., 2min; (94° C., 30 sec; 55° C., 30 sec; 72° C., 60 sec)×25cycles; 4° C. hold.

The PCR product was run on a 1.5% agarose gel. A specific band of the expected size was isolated. The PCR-product and the vector pENI2149 were cut with restriction enzymes (BamHI/NotI). The vector and the PCR product were run on a 1% agarose gel, and purified from the gel. The cut PCR-product and the cut vector were mixed in a ligase buffer with T4 DNA ligase (Promega). After overnight ligation at 16° C., the mixture was transformed into E. coil strain DH10B. The laccase gene of 3 randomly picked transformants were sequenced to assess whether or not the mutagenic primers had been incorporated during the course of the assembly reactions (Table1):

TABLE 1

| | Primer | | | | | | | | | |
|---|---|---|---|---|---|---|---|---|---|---|
| | La2 | La3 | La4 | La5 | La6 | La7 | La8 | La9 | La10 | La11 |
| | Clone | | | | | | | | | |
| | H91Q | H91R | H133Q | H133R | H153Q | H153Q | H230Q | H230R | H309Q | H309R |
| A1 | x | | | x | | | | x | x | |
| A2 | | | | x | | | | | | |
| A3 | x | | x | | x | | | x | x | |

### EXAMPLE 2

### Gene Shufflinq Using DNA Degraded by Piperidine

The gene encoding the haloperoxidase from Curvularia verruculosa was cloned into the E. coli expression vector pSE420 to generate plasmid pSE420-CvHAP. Using this plasmid as template, two PCR fragments were generated using primers:

| | | |
|---|---|---|
| a) | SEQ ID NO: 15 | 5′-gtttcccgactggaaagcgggcagtg + |
| | SEQ ID NO: 16 | 5′-caccgatagggaagaggccctcg |
| | and | |
| b) | SEQ ID NO: 17 | 5′-gagagtcagtcagcttcatgt + |
| | SEQ ID NO: 18 | 5′-gcttctgcgttctgatttaatc |

PCR was performed using 0.5 unit Amplitaq® DNA polymerase, suppliers buffer, 250 μM each of dATP, dCTP and dGTP, 200 μM of dTTP, and 50 μM of dUTP.

PCR cycling was as follows: 94° C., 2min; (94° C., 30 sec; 55° C., 30 sec; 72° C., 2 min)×25 cycles; 72° C., 5 minutes; 4° C. hold. DNA was purified on a PCR-purification column (Qiagen), and eluted into 100 μl 10 mM Tris-HCl, pH 7.5. 20 μl (20 units) UDG (NEB), 14 μl NE buffer 4 and 6 μl DpnI (NEB) were added, and the tube was incubated at 37° C. for 16 hrs. Thereafter DNA was precipitated by addition of 1/10 vol 3M NaAc, 2.5 vol 96% EtOH. Precipitated DNA was washed by 70% EtOH, dried and dissolved in freshly prepared 1M piperidine. The tube was placed at 90° C. for 20 min and thereafter transferred to a vacuum desiccator to evaporate the piperidine solution. Dried fragmented DNA was dissolved in 50 μl 10 mM Tris-HCl and 10 μl (approximately 1 μg) was used for a PCR assembly reaction in a total volume of 50 μl using 0.5 unit Pwo polymerase, suppliers buffer and 250 μM dNTP's.

PCR cycling was as follows: 94° C., 2min; (94° C., 30 sec; 48° C., 30 sec; 72° C., 1 min)×30 cycles, 720° C., 5 minutes, 40° C. hold. 10 μl of the PCR products were run on a 1% agarose gel. A smear of DNA was seen between 400 and 2000 bp. Half of the PCR product was mixed in a tube with two specific primers (50 pmol) flanking the gene of interest, 250 μM dNTP, 5 μl 10× Taq buffer, 2.5 mM MgCl₂.

Then the following standard PCR-program was run: (94° C., 5 minutes) 1 cycle; (94° C. 30 seconds; 50° C., 30 seconds; 72° C. 60 seconds)×25 cycles; 72° C., 7 minutes; 4° C., hold. The PCR product was run on a 1.5% agarose gel. A specific band of the expected size was isolated. The PCR-product and the vector pSE420 were cut with restriction enzymes (NcoI/NotI). The vector and the PCR product were run on a 1.5% agarose gel, and purified from the gel. The cut PCR-product and the cut vector were mixed in a ligase buffer with T4 DNA ligase (Promega). After overnight ligation at 16° C. the mixture was transformed into E. coli strain DH10B, and 2 independent transformants were sequenced to verify that the entire haloperoxidase had been reassembled.

### EXAMPLE 3

### Construction of a Library of Enzyme Variants Using Deuracilated, Non-Degraded, DNA

A genomic fragment of the laccase from Coprinus cinereus was inserted into A. oryzae expression vector pENI2149 to create plasmid pCC2. 20 ng of this plasmid was used as template for PCR in a total volume of 100 μl using 1 μM each of primers:

| | |
|---|---|
| SEQ ID NO: 19 | 5′-agggatgccatgcttggagtttcc |
| and | |
| SEQ ID NO: 20 | 5′-ccaattgccctcatccccatcc |

PCR was performed using 0.5 units Amplitaq® DNA polymerase, suppliers buffer, 250 μM each of dATP, dCTP, and dGTP, 200 μM of dTTP, and 50 μM of dUTP. PCR cycling was as follows: 94° C., 2min; (94° C., 30 sec; 55° C., 30 sec; 72° C., 2 min)×25 cycles; 72° C., 5 minutes; 4° C. hold. PCR products were purifed on a PCR-purification column (Qiagen), and eluted into 100 μl 10 mM Tris-HCl, pH 7.5. 20 μl (20 units) UDG (NEB), 14 μl NE buffer 4 and 6 μl DpnI (NEB) were added and the tube was incubated at 37° C. for 16 hrs. Following this treatment the DNA is gel purified and the deuracilated DNA used as a template in a series of experiments with different numbers and concentrations of mutagenic primers. PCR cycling should be performed in a total volume of 50 μl using Pwo polymerase, suppliers buffer and 250 μM dNTP's.

PCR cycling can be performed as follows: 94° C., 2min; (94° C., 30 sec; 48° C., 30 sec; 72° C., 3 sec)×30 cycles; 72° C., 5 minutes; 4° C. hold. Half of the PCR product is then mixed in a tube with two specific primers (50 pmol) flanking the gene of interest, 250 μM dNTP, 5 μl 10× Taq buffer, 2.5 mM MgCl₂ and H₂O to 50 μl. The following standard PCR-program is run: (94° C. , 5 minutes) 1 cycle; (94° C., 30 seconds; 50° C., 30 seconds; 72° C., 60 seconds)×25 cycles; 72° C., 7 minutes; 4° C., hold. The PCR product can be run on a 1.5% agarose gel, and a specific band of the expected size isolated and cloned into an appropriate expression vector.

1. A method for producing recombined polynucleotides, the method comprising the steps of:
i) providing a polynucleotide population comprising one or more nucleotide(s) or nucleotide analogue(s) different from dATP, dCTP, dGTP, and dTTP;
ii) excising the base-moiety of said nucleotide(s) or nucleotide-analogue(s) from the polynucleotide population of i) under conditions which promote cleavage of sugar-base bonds in polynucleotides, thereby generating one or more AP-site(s) in the polynucleotide population;
iii) annealing at least one primer to the polynucleotide population of ii) and extending the primer(s) by polynucleotide synthesis;
iv) dissociating the extended primer(s) of step iii) and the polynucleotide population, reannealing the extended primers to the polynucleotide population and further extending the primer(s) by polynucleotide syntesis; and optionally
v) repeating step iv) one or more times.

2. A method for producing recombined polynucleotides, the method comprising the steps of:
i) providing a polynucleotide population comprising one or more nucleotide(s) or nucleotide analogue(s) different from dATP, dCTP, dGTP, and dTTP;
ii) excising the base-moiety of said nucleotide(s) or nucleotide-analogue(s) from the polynucleotide population of i) under conditions which promote cleavage of sugar-base bonds in polynucleotides, thereby generating one or more AP-site(s) in the polynucleotide population;
iii) cleaving the polynucleotide population of ii) at said AP-site(s);
iv) annealing at least one primer to the polynucleotide population of iii) and extending the primer(s) by polynucleotide synthesis;
v) dissociating the extended primer(s) of step iii) and the polynucleotide population, reannealing the extended primers to the polynucleotide population and further extending the primer(s) by polynucleotide syntesis; and optionally
vi) repeating step v) one or more times.

3. The method of claim 1 or 2, wherein the polynucleotide population of step i) or the primer extending is provided by performing a polymerase chain reaction with at least one DNA polymerase or with a mixture of at least two DNA polymerases, preferably with one or more DNA polymerase(s) chosen from the group consisting of: Taq-polymerase, Amplitaq®-polymerase, Vent®-polymerase, Pwo-polymerase, Pfu-polymerase, Tth-polymerase, T4 polymerase, T7 polymerase, E. coli DNA-polymerase I, Stoffel fragment, and Klenow fragment of DNA-polymerase I.

4. The method of claim 1 or 2, wherein the polynucleotide population of step i) is isolated from a host cell which is capable of incorporating nucleotide(s) or nucleotide analogue(s) different from dATP, dCTP, dGTP, and dTTP into a polynucleotide during polynucleotide replication or in vivo synthesis.

5. The method of claim 1 or 2, wherein the polynucleotide population of step i) is provided by chemical synthesis.

6. The method of any of claims 1-5, wherein said primer(s) comprises one or more random or semi-random primers.

7. The method of any of claims 1-5, wherein said primer(s) comprises one or more mutagenic primers.

8. The method of any of claims 1-5, wherein said primer(s) comprises one or more specific primers.

9. The method of any of claims 1-8, wherein said nucleotide(s) or nucleotide analogue(s) comprises dUTP, 5-fluoro-dUTP, dITP, 3-methyl-dATP, 7-methyl-dATP, 7-methyl-dGTP, or a mixture of these.

10. The method of any of the claims 1-9, wherein the rate, in the polynucleotide population of step i) of each nucleotide or nucleotide analogue that is different from dATP, dCTP, dGTP, and dTTP to the corresponding naturally occurring nucleotide(s), is controlled by optimizing the ratio of said nucleotide(s) or nucleotide analogue(s) to the corresponding naturally occurring nucleotide(s) during synthesis of the polynucleotide population of step i).

11. The method of claim 10, wherein the nucleotide dUTP is used and the dUTP/dTTP ratio is about 0.02-1.5.

12. The method of claim 11, wherein the dUTP/dTTP ratio is about 0.1-0.8.

13. The method of any of claims 1-12, wherein excising the base-moiety of said nucleotide(s) or nucleotide-analogue(s) from the polynucleotide population is done by using a DNA glycosylase (EC 3.2.2.-) suitable for cleaving the base-moiety of the nucleotide(s) or nucleotide analogue(s) comprised in the polynucleotide population of step i).

14. The method of claim 13, wherein the DNA-glycosylase is an uracil-DNA glycosylase, a hypoxanthine-DNA glycosylase, a 3-methyladenine-DNA glycosylase I, a 3-methyladenine-DNA glycosylase II, a formamidopyrimidine-DNA glycosylase, or a mixture of these.

15. The method of any of claims 2-14, wherein the cleaving at the AP-site(s) is done by using one or more AP-endonuclease(s), preferably an AP-endonuclease chosen from the group consisting of Escherichia coli exonuclease III, E. coli endonuclease IV, and E. coli endonuclease V; or a mammalian AP endonuclease; or a mixture of these.

16. The method of any of claims 2-14, wherein the cleaving at the AP-site(s) is done by using piperidine.

17. The method of any of claims 2-14, wherein the cleaving at the AP-site(s) is done by increasing the temperature and/or alkaline conditions, preferably with a pH of at least 8.

18. The method of any of claims 1-17, wherein the polynucleotide population of step i) comprises mutants or variants of the same native polynucleotide, or comprises homologous polynucleotides isolated from nature, or both.

19. The method of any of claims 1-18, wherein at least one individual polynucleotide of the population of step i) exhibits a nucleotide sequence %-identity of at least 50%, preferably 60%, more preferably 70%, still more preferably 80%, even more preferably 90%, or most preferably at least 95% to at least one other polynucleotide of the population.

20. The method of any of claims 1-19, wherein the polynucleotide population of step i) originates from at least two wild type organisms of different genera or preferably from different species.

21. The method of any of claims 1-20, wherein said polynucleotide population of step i) is cloned into a suitable vector, preferably the vector is a plasmid.

22. The method of any of claims 1-21, wherein the polynucleotide population of step i) comprises polynucleotides encoding at least one enzyme, preferably at least a hydrolase, a lyase, a ligase, a transferase, an isomerase, or an oxidoreductase.

23. The method of any of claims 1-21, wherein the polynucleotide population of step i) comprises polynucleotides encoding at least one polypeptide or peptide having antimicrobial activity.

24. The method of any of claims 1-21, wherein the polynucleotide population of step i) comprises at least one polynucleotide encoding a polypeptide having biological activity; preferably the polypeptide is insulin, pro-insulin, pre-pro-insulin, glucagon, somatostatin, somatotropin, thymosin, parathyroid hormone, pituitary hormones, somatomedin, erythro-poietin, luteinizing hormone, chorionic gonadotropin, hypothalamic releasing factor, antidiuretic hormone, blood coagulant factor, thyroid stimulating hormone, relaxin, interferon, thrombopoeitin (TPO) or prolactin.

25. The method of any of claims 1-21, wherein the polynucleotide population of step i) comprises at least one polynucleotide which has a biological function, preferably in transcription initiation or termination, translational initiation, or as an operator site related to expression of one or more gene(s).

26. A method for producing recombined polynucleotides, the method comprising the steps of providing a polynucleotide population comprising one or more nucleotide(s) or nucleotide analogue(s) different from dATP, dCTP, dGTP, and dTTP, wherein said nucleotide(s) or nucleotide analogue(s) are suitable as targets for polynucleotide strand cleavage, cleaving said strands, and recombining and extending the products by polynucleotide synthesis.

27. A method for using recombined polynucleotides obtained by a method as defined in any of the claims 1-26 in identifying an encoded polypeptide having an activity of interest, where the polypeptide exhibits at least one altered property in comparison to known polypeptides that have the same activity, wherein said recombined polynucleotides are cloned into an appropriate vector, said vector is transformed into a suitable host cell wherein said encoded polypeptides are expressed, the polypeptides are screened in a suitable assay, an altered polypeptide of interest is identified, and the vector comprising the encoding polynucleotide is isolated.

28. A method for producing a polypeptide of Interest as defined in claim 27, wherein the polynucleotide encoding the polypeptide of interest is cloned into a suitable expression vector and transformed into a suitable host cell which is cultivated under conditions suitable for expression of said polypeptide, and optionally the polypeptide is recovered.
